(19) United States
(12) Patent Application Publication
(10) Pub. No.: US 2005/0228226 A1
(43) Pub. Date: Oct. 13, 2005

(54) Video endoscope fitted with a terminal part, and a terminal part for a video endoscope

(75) Inventor: Andreas Muckner, Hamburg (DE)

Correspondence Address: RANKIN, HILL, PORTER & CLARK LLP, 4080 ERIE STREET, WILLOUGHBY, OH, 44094-7836, US

(73) Assignee: OLYMPUS WINTER & IBE GMBH, Hamburg (DE)

(21) Appl. No.: 11/100,723

(22) Filed: Apr. 7, 2005

(30) Foreign Application Priority Data
Apr. 8, 2004 (DE) 102004018123.3

(51) Int. Cl. A61B 1/05

(52) U.S. Cl. 600/110; 600/130; 600/134

(57) ABSTRACT

A rigid video endoscope, fitted with a tube unit (1) comprising a metallic outer tube (2, 2′) sealed off distally by a window (3) and a metallic inner tube (4, 4′) which supports a video camera (5) and an objective (6) in its distal end segment (4), said tube unit being fitted with a proximal terminal part (12) wherein the proximal end segment (4′) of the inner tube (4, 4′) is sealed off by a glass fusion element (7) that is traversed by fusion-imbedded metal contact pins (8) of which the inward ends are connected by conductors (10) to contact pins (9) of the video camera (5) and wherein the proximal end segment (2′) of the outer tube (2, 2′) is hermetically connected to the proximal end segment (4′) of the inner tube (4, 4′), the outer tube (2, 2′) being electrically insulated at least at its distal end segment (2) from the inner tube (2, 2′), is characterized in that the proximal end segments (2′, 4′) of the two tubes (2, 2′; 4, 4′) are connected in the terminal part (12) by means of an annular glass fusion element (11),

### BACKGROUND OF THE INVENTION

1. Field of the Invention

The present invention relates to a video endoscope and moreover to a terminal part for the video endoscope.

2. Description of Related Art

Video endoscopes of this kind comprise a tube unit including an outer tube fitted with a distal window and hermetically sealed relative to the ambient for the purpose to prevent liquid and vapor from entering the inner space for instance when autoclaving. An inner tube is configured within the outer tube and supports an objective-fitted camera situated in front of the window. The proximal inner tube end is hermetically sealed by a glass fusion element crossed by contact pins serving as electrical terminals to the camera in the inner tube and acting as plug-in connectors on the outside.

For electrical safety, the metallic outer tube must be electrically insulated relative to the inner tube in order to prevent it from making external electrical contact with the inner tube that might carry a current.

In one design of the above species used by applicant, the outer tube is split, with a proximal segment being metallically connected to the inner tube and being linked by an insulating ring. As a result, at least the distal outer tube segment shall be adequately insulated. However this design incurs hermeticity problems and its manufacture is complex.

### SUMMARY OF THE INVENTION

The objective of the present invention is to improve and simplify the design of the video endoscope of the above species as regards hermeticity and electrical safety.

In the present invention, the proximal terminal part of the inner tube is linked in the end segment of the tube unit by an annular glass fusion element to the proximal terminal part of the outer tube. Such a fusion glass element offers the advantage of absolute hermeticity, in particular with respect to vapors, namely it offers this advantage also with respect to conventional autoclaving. Moreover the same technique of glass fusion elements is also applied to sealing the inner tube inward and outward relative to the outer tube, whereby manufacture is substantially simplified and made more economical. Said glass fusion element also provides excellent electrical insulation, hence the entire outer tuber including its proximal terminal part is electrically insulated from the inner tube. The segments of the outer tube may be connected in simple manner metallically, or they be made integral with one another.

Continuous outer tubes and inner tubes also may be linked to each other in the manner of the invention. However, the terminal part is designed to comprise the proximal end segments of the two tubes and the two glass fusion elements is a prefabricated part which may be linked to the proximal end segments of the two tubes. In this manner manufacturing costs may be once more lowered drastically. The terminal part with the two glass fusion zones may be economically prefabricated in a specialty shop outfitted for that purpose and then may be linked to the remaining tube lengths of the two tubes in conventionally manner, for instance by laser welding, to provide highly hermetic seal. As a result a modular design with large manufacturing advantages is attained.

### BRIEF DESCRIPTION OF THE DRAWING

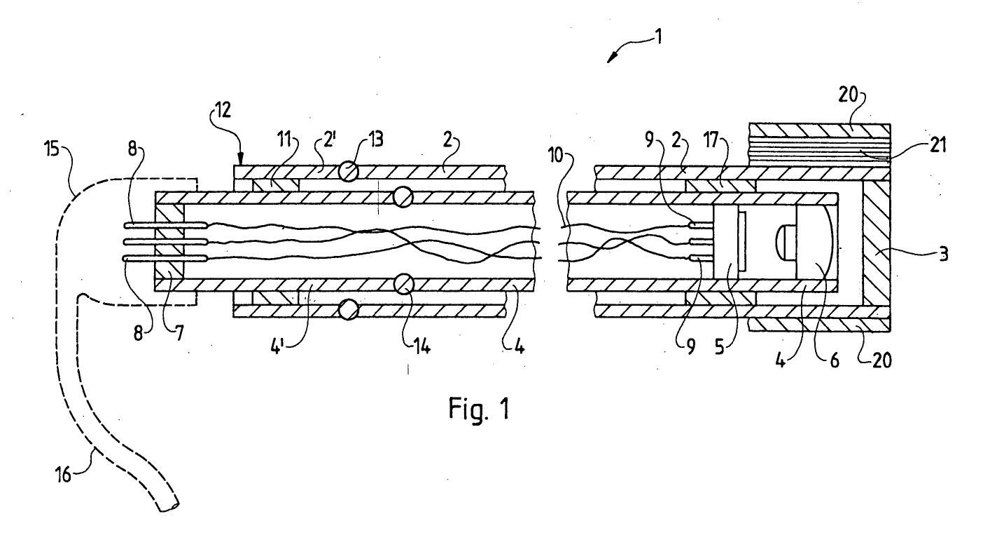

The appended drawing elucidates the invention in illustrative and schematic manner in a single FIG. 1 representing an axial section of a video endoscope of the invention.

### DETAILED DESCRIPTION OF THE INVENTION

FIG. 1 is an axial section of a rigid video endoscope of the invention comprising a tube unit 1. Said unit 1 in turn comprises a metallic outer tube 2 fitted with a proximal end segment 2′ and a distal end segment 2 sealed off by a window 3, illustratively hermetically by a rim solder seal. An inner tube 4, 4′ is configured within the outer tube 2, 2′ and also is made of a suitable metal, a video camera 5 of appropriate design fitted with an associated objective 6 being configured in the distal end segment 4 of said inner tube.

The inner tube is sealed off at its proximal end segment 4′ by a glass fusion element 7 which is traversed by fusion-imbedded contact pins 8. The video camera 5 is fitted with contact pins 9. Electric hookup conductors 10 making contact at both ends run between the contact pins 8 and 9.

The proximal end segments 4′, 2′ of the two tubes are rigidly joined by glass fusion ring 11 and are sealed off relative to each other. Accordingly the inner space of the outer tube 2, 2′ sealed in fully hermetic manner by the gas-tight window 3 and by the two gas fusion elements 7 and 11.

In the illustrated and preferred embodiment mode of FIG. 1, the proximal end segments 2′, 4′ of the two tubes of the tube unit 1 constitute a proximal terminal part 12.

The proximal end segment 2′ of the outer tube is connected to said tube's distal end segment 2 by an annular welding bead 13 and the proximal end segment 4′ of the inner tube is connected said tube's end segment 4 by an annular welding bead 14. Preferably the two welding beads are made into highly hermetic junctions by laser welding.

When assembling the said endoscope, first the terminal part 12 is prefabricated by glass melting techniques. Next the conductors 10 are soldered to the contact pins 8 and 9. Thereupon the welding beads 13 and 14 are applied to the assembly.

In the finished tube unit 1, the terminal part 12 may act as a connector element for an electrical socket 15 shown in dashed lines in FIG. 1 and making electrical contact by means of omitted contacts with the outer ends of the contact pins 8 and by means of a cable 16 implementing connection to an omitted video processing unit.

Instead of the embodiment mode shown above, which comprises a prefabricated terminal part 12, the design of the invention also may be integral, that is omitting the welding beads 13 and 14, namely using a continuous outer tube 2, 2′ and inner tube 4, 4′.

In the design of FIG. 1, the affixation between the outer tube 2, 2′ and the inner tube 4, 4′ is implemented by the annular glass fusion element 11. Additional bracing between the distal end segments 2, 4 of the two tubes may be provided in the form of a spacer ring 17 which is inserted in clamping or another affixing manner between the two tubes. The spacer ring 17 also preferably is made of an electrically insulating material, preferably plastic or ceramic.

As shown by FIG. 1, the tube unit 1 is mounted within an outer stem 20, optic fibers 21 being configured between this outer stem and the outer tube 2, 2′. As further shown by FIG. 1, the outer tube 2, 2′ and the outer stem 20 in some places and circumstances might make metallic contact. However, because of the electrical insulation between the outer tube 2, 2′ and the inner tube 4, 4′, the outer stem 20—which typically is made of metal—remains electrically safe if for instance one or more of the conductors 10 should electrically touch the inner tube 4, 4′ because of defective installation.

1. A rigid video endoscope fitted with a tube unit (1) which comprises a metallic outer tube (2, 2′) distally closed by a window (3) and a metallic inner tube (4, 4′) supporting in its distal end segment (4) a video camera (5) and an objective (6), where the tube unit (1) comprises a proximal terminal part (12) wherein the end segment (4′) of the inner tube (4, 4′) is sealed off by a glass fusion element (7) that is traversed by fusion-imbedded metallic contact pins (8) of which the inward ends are connected by conductors (10) to terminals (9) of the video camera (5) and wherein the proximal end segment (2′) of the outer tube (2, 2′) is hermetically connected to the proximal end segment (4′) of the inner tube (4, 4′), the outer tube (2, 2′) being mounted at least by its distal end segment (2) in electrically insulated manner relative to the inner tube (4, 4′) characterized in that
the proximal end segments (2′, 4′) of the two tubes (2, 2; 4, 4′) are connected by an annular glass fusion element (11).

2. Apparatus as claimed in claim 1, characterized in that the terminal part (12) is prefabricated together with the distal end segments (2′, 4′) of the two tubes (2, 2′; 4, 4′) and the two glass fusion elements (7, 11) and is connectable to the proximal end segments (2, 4) of the two tubes.

3. A terminal part for rigid video endoscopes as claimed in claim 2.
